SEQUENCE LISTING (3 sequences)

SEQ ID NO: 1 - DNA, 25 bp, Artificial Sequence
  Forward primer sequence
ggagaaagtcacagggacacagttt

SEQ ID NO: 2 - DNA, 24 bp, Artificial Sequence
  Reverse primer sequence
ggaaacccatgacagaggcatata

SEQ ID NO: 3 - DNA, 28 bp, Artificial sequence
  Probe sequence
ccaaatgatgtcagccatgggtatgtga

(19) United States
(12) Patent Application Publication
(10) Pub. No.: US 2009/0082402 A1
(43) Pub. Date: Mar. 26, 2009

(54) Methods of treating genitourinary disorders using inhibitors of soluble epoxide hydrolase

(75) Inventors: Preston Albert Baecker, Los Altos, CA (US); Thomas Chang, Atherton, CA (US); Alan H. Kosaka, Half Moon Bay, CA (US); Joseph S. Cefalu, San Jose, CA (US); Philip A. Nunn, Los Altos, CA (US)

Correspondence Address: ROCHE PALO ALTO LLC;PATENT LAW DEPT. M/S A2-250, 3431 HILLVIEW AVENUE, PALO ALTO, CA, 94304, US

(73) Assignee: Roche Palo Alto LLC

(21) Appl. No.: 12/002,646

(22) Filed: Dec. 18, 2007

Related U.S. Application Data
(60) Provisional application No. 60/875,848, filed on Dec. 18, 2006.

(51) Int. Cl. A61K 31/444; G01N 33/53; C12Q 1/34

(52) U.S. Cl. 514/333; 435/7.8; 435/18

(57) ABSTRACT

The invention relates to methods of treating or preventing a disease state associated with a genitourinary disorder using inhibitors of soluble epoxide hydrolase.

### CROSS REFERENCE TO RELATED INVENTIONS

This application claims the benefit of priority of U.S. Provisional Patent Application Ser. No. 60/875,848 filed Dec. 18, 2006, which is incorporated herein by reference in its entirety.

### FIELD OF THE INVENTION

The present invention relates generally to the discovery that inhibitors of soluble epoxide hydrolase can be useful for the treatment of a disease state associated with a genitourinary disorder. In particular the present invention relates to methods of treating or preventing a disease state associated with a genitourinary disorder using inhibitors of soluble epoxide hydrolase.

### BACKGROUND OF THE INVENTION

Epoxide hydrolases are a group of enzymes that catalyze the addition of water to an epoxide, resulting in a vicinal diol (Hammock et al (1997) in Comprehensive Toxicology: Biotransformation (Elsevier, N.Y.), pp. 283-305). Epoxide hydrolase was first studied in insects in the context of juvenile hormone biosynthesis (Gill et al (1972) in Insect Juvenile Hormones: Chemistry and Action, eds. J. J. Menn and M Beroza (Academic Press, New York), pp. 177-189), but is now known to occur widely in nature. Several types of epoxide hydrolases have been characterized in mammals including soluble epoxide hydrolase (sEH), also known as cytosolic epoxide hydrolase, cholesterol epoxide hydrolase, LTA₄ hydrolase, hepoxilin epoxide hydrolase and microsomal epoxide hydrolase (Fretland and Omiecinski, Chemico-Biological Interactions, 129: 4159 (2000)). Epoxide hydrolases have been found in a variety of tissues in vertebrates including heart, kidney and liver (Vogel, et al., Eur J Biochemistry, 126: 425-431 (1982); Schladt et al., Biochem. Pharmacol., 35: 3309-3316 (1986)).

The known endogenous substrates of sEH are four epoxy regioisomers of arachidonic acid known as epoxyeicosatrienoic acids or EETs. These are 5,6-, 8,9-, 11,12-, and 14,15-epoxyeicosatrienoic acid. The products generated by hydrolysis of these substrates are the dihydroxyeicosatrienoic acids or DHETS, 5,6-, 8,9-, 11,12-, and 14,15-dihydroxyeicosatrienoic acid respectively. Also known to be substrates are epoxides of linoleic acid known as leukotoxin or isoleukotoxin. Both the EETs and the leukotoxins are generated by members of the cytochrome P450 monooxygenase family (Capdevila, et al., J. Lipid Res., 41: 163-181 (2000)). The structural requirements for substrates of sEH have recently been described (Morisseau et al., Biochem. Pharmacol. 63:1599-1608 (2002)) and the crystal structure, as well as structures of co-crystals with inhibitors determined (Argiriadi et al., Proc. Natl. Acad. Sci. USA 96: 10637-10642 (1999)). A variety of inhibitors of sEH have also been described (Mullin and Hammock, Arch. Biochem. Biophys. 216:423-439 (1982), Morisseau et al., Proc. Natl. Acad. Sci. USA 96:8849-8854 (1999), McElroy et al., J. Med. Chem. 46:1066-1080 (2003)). A phosphatase activity for phosphorylated forms of hydroxy unsaturated fatty acids has recently been described for soluble epoxide hydrolase, making this a bifunctional enzyme (Newman, et al., Proc. Natl. Acad. Sci. USA 100:1558-1563 (2003)). The physiological significance of this phosphatase activity has not been established.

The physiological role of EETs has best been established in vasodilation of vascular beds. Evidence has accumulated that EETs in fact function as endothelium-derived hyperpolarizing factors or EDHFs (Campbell et al., Circ. Res. 78:415-423 (1996)). EETs are formed in endothelial cells, induce vasodilation in vascular smooth muscle cells by a mechanism that results in activation of “maxi K” potassium channels with attendant hyperpolarization and relaxation (Hu and Kim, Eur. J. Pharmacol. 230:215-221)). It has been shown that 14,15-EET exerts its physiological effects by binding to cell surface receptors that are regulated by intracellular cyclic AMP and by a signal transduction mechanism involving protein kinase A (Wong et al., J. Lipid Med. Cell Signal. 16, 155-169 (1997)). More recently, this EET dependent relaxation in coronary smooth muscle was demonstrated to occur through a guanine nucleotide binding protein, G₅α, accompanied by ADP-ribosylation (Li et al., Circ. Res. 85, 349-56 (1999). Alternatively, the cation channel TRPV4, has recently been shown to be activated by 5,6-EET in mouse aorta vascular endothelial cells (Watanabe et al., Nature 424, 434-438 (2003)). This has generated interest in EETs and soluble epoxide hydrolase as targets for antihypertensives. Indeed, male sEH knockout mice have reduced blood pressure as compared to wild type controls (Sinal et al., J. Biol. Chem. 275:40504-40510 (2000)). Furthermore, inhibition of sEH in spontaneously hypertensive rats caused a reduction in blood pressure (Yu et al., Circ. Res. 87:992-998 (2000)).

EETs, especially 11,12-EET, also have been shown to exhibit anti-inflammatory properties (Node, et al., Science, 285:1276-1279 (1999); Zeldin and Liao, TIPS, 21: 127-128 (2000)). Node, et al. have demonstrated 11,12-EET decreases expression of cytokine induced endothelial cell adhesion molecules, especially VCAM-1. They further showed that EETs prevent leukocyte adhesion to the vascular wall and that the mechanism responsible involves inhibition of NF-κB and iκB kinase. Although inhibitors of sEH are useful for the treatment of cardiovascular and inflammatory diseases, it has not been previously discovered that inhibitors of sEH can be useful for the treatment of genitourinary diseases.

The spontaneously hypertensive rat (SHR) is an animal model for hypertension derived from selective breeding of Wistar-Kyoto (WKY) rats for elevated blood pressure. The SHR also shows increased urinary frequency, voiding about three times more frequently than Wistar-Kyoto controls during waking hours (Clemow, D. et al Neurourol Urodyn. 16, 293 (1997)). A variety of proposals have been advanced for the etiology behind the hyperactive voiding in SHR, but the variety of proposals in themselves indicate a lack of compelling evidence. One study shows that backcrosses of F1 generation SHR X WKY hybrids, results in a high correlation between inheritance of the frequent voiding phenotype and the hypertensive phenotype (Clemow et al, J. Urol. 161:1372-1377 (1999)), suggesting that some genetic determinants might be common to both phenotypes.

A number of studies in SHR have shown mapping of loci that correlate with hypertension. At one time CD36, a fatty acid transport protein, appeared to be an excellent candidate that was essentially absent in domestic colonies of SHR, and when transgenically added back into SHR stock, reversed the hypertensive phenotype (Aitman, et al., Nat. Genet. 21:76-83 (1999)). Later, it was pointed out that contrary to these encouraging results, the original colony of SHR in Japan expressed CD36 normally despite their hypertensive phenotype effectively excluding CD36 as a defining mutation (Gotoda et al., Nat. Genet. 22:226-228 (1999)). More recently, genetic linkage studies in the F2 generation of other backcrosses between hypertensive and normotensive rat strains has shown loci on chromosomes 2 and 10 that contribute to hypertension. The chromosome 2 locus appears to be for the Npr1 gene encoding a member of the natriuretic peptide receptor family while the chromosome 10 locus contains the Ace gene. The effect of these loci on voiding has not been reported.

Soluble epoxide hydrolase has been reported to be elevated in some tissues in SHR (although there are no indications of elevation in bladder) and the amount of sEH found in SHR has been reported to be variable dependent on the source of the animals (Okuda et al., Biochem. Biophys. Res. Comm. 296:537-543 (2002)). This is a frequent observation with expression of specific genes in SHR. There is a higher degree of genetic heterogeneity in SHR than is usually the case with inbred strains of rodents and the genetic makeup of any given colony may differ from that of other colonies, depending on the genetic composition of the founder pairs (Nabika et al., Hypertension 18:12-16 (1991)). Cytochrome P450 2J14 which is in part responsible for epoxidation of arachidonic acid to 14,15-EET, has been shown to be specifically elevated in SHR among several cytochrome P450s (Yu et al., Mol. Pharmacol. 57:1011-1020 (2000)). It is not clear whether sEH is elevated as a consequence of CYP2J14 elevation or vice versa. Alternatively, both may be elevated as a consequence of a perturbation in a signaling pathway that is yet to be elucidated.

Urinary incontinence can be roughly categorized into four main classes: 1) Urge incontinence associated with bladder instability; 2) Stress incontinence associated with a weak bladder neck/urethral function; 3) Mixed incontinence in which mechanisms for both urge and stress occur together; 4) overflow incontinence due to mechanical obstruction or functional disorders. In urge incontinence, the most common form subjected to medical treatment, several mechanism may be involved in the pathogenesis of the disease including myogenic or neurological (MS, stroke, Parkinson disease, spinal cord injury) factors. The condition is characterized by frequent abnormal detrusor contractions associated with the involuntary leakage of urine and urgency.

The most widely used therapeutics for this condition are the antimuscarinics oxybutynin and tolterodine, which work via inhibiting the smooth muscle contractility and reducing basal bladder tone, however their utility is limited by their class side effect profile including dry mouth, constipation and cognition impairment.

The present invention shows promise for treatment of incontinence by intervention in these abnormal detrusor contractions without the side effects associated with antimuscarinics.

### SUMMARY OF THE INVENTION

The present invention provides a method of treating a mammalian subject having a disease state associated with a genitourinary disorder comprising administering to the subject an effective amount of an inhibitor of soluble epoxide hydrolase. In a further embodiment the genitourinary disorder is an overactive bladder, outlet obstruction, outlet insufficiency, interstitial cystitis, male erectile dysfunction, or pelvic hypersensitivity. In another embodiment, the effective amount of the soluble epoxide hydrolase inhibitor is administered orally. Preferably, the soluble epoxide hydrolase inhibitor has an IC50 of less than 1 μM. In a further embodiment, the mammalian subject is a human.

The present invention provides a method for decreasing the frequency and amplitude of bladder contraction in a mammalian subject comprising administering to the subject an effective amount of an inhibitor of soluble epoxide hydrolase. The present invention also provides a method of identifying compounds that decrease the frequency and amplitude of bladder contraction, comprising contacting the compound with soluble epoxide hydrolase and determining whether the compound inhibits soluble epoxide hydrolase and testing the compound in a functional assay that measures the effect of the compound on bladder contraction frequency and amplitude.

The present invention further provides a method of identifying a mammalian subject at risk for a genitourinary disorder comprising assaying for soluble epoxide hydrolase level or activity (or balance between substrates and products) in a sample from the subject, preferably a urine sample or a bladder tissue.

The present invention further provides a method of treating a mammalian subject having a disease state associated with a genitourinary disorder comprising administering to the subject an effective amount of a 14,15-EET receptor agonist, preferably where the agonist has an affinity value of less than 100 nM to the 14,15-EET receptor.

### BRIEF DESCRIPTION OF THE DRAWINGS

FIG. 1 is a cystometry tracing of the effects of Compound 1 on bladder pressure in anesthetized SHRs.

FIG. 2 shows the effects of Compound 1 on Mean Blood Pressure (MBP), Bladder Contraction Frequency (Frequency) and Bladder Contraction Amplitude compared to vehicle in SHRs.

### DETAILED DESCRIPTION OF THE INVENTION

### Definitions

Unless otherwise stated, the following terms used in this Application, including the specification and claims, have the definitions given below. It must be noted that, as used in the specification and the appended claims, the singular forms “a”, “an,” and “the” include plural referents unless the context clearly dictates otherwise.

The term “14,15-EET receptor agonist” refers to a molecule which, when bound to the 14,15-EET receptor, or is within proximity of the 14,15-EET receptor, modulates the activity of such receptor by increasing or prolonging the duration of the effect of the receptor. Agonists can include 14,15-EET and other epoxyeicosatrienoic acids as well as nucleotides, proteins, nucleic acids, carbohydrates, organic compounds, inorganic compounds, or any other molecules which modulate the effect of the 14,15-EET receptor.

The term “disease state” refers to any disease, condition, symptom, disorder, or indication.

The term “disease state associated with a genitourinary disorder”, which is used interchangeably with “symptoms associated with a genitourinary disorder”, refers to disease states associated with the urinary tract including, but not limited to, overactive bladder, outlet obstruction, outlet insufficiency, benign prostatic hyperplasia, interstitial cystitis, male erectile dysfunction and pelvic hypersensitivity. In particular, the compounds of the present invention may be useful in the treatment of symptoms associated with the above disease state, e.g., urgency, frequency, altered bladder capacity, incontinence, micturition threshold, unstable bladder contractions, sphincteric spasticity, detrusor hyperreflexia (neurogenic bladder), detrusor instability, benign prostatic hyperplasia (BPH), urethral stricture disease, tumors, low flow rates, difficulty in initiating urination, urgency, suprapubic pain, urethral hypermobility, intrinsic sphincteric deficiency, mixed incontinence, stress incontinence, pelvic pain, interstitial (cell) cystitis, prostadynia, prostatis, vulvadynia, urethritis, orchidalgia, and other symptoms related to overactive bladder.

The terms “effective amount” or “therapeutically effective amount” refer to a nontoxic but sufficient amount of the agent to provide the desired biological result. That result can be reduction and/or alleviation of the signs, symptoms, or causes of a disease, or any other desired alteration of a biological system. An appropriate “effective” amount in any individual case may be determined by one of ordinary skill in the art using routine experimentation.

The term “interstitial cystitis” refers to a chronic inflammatory condition of the bladder wall of unknown cause or causes displaying symptoms of urinary urgency and frequency, difficulty in urinating, small urine output, and pain in the bladder and/or urethra that is temporarily relieved by voiding. In some cases, pain may radiate to the genitals, rectal area and thighs. Cystoscopic examination of the bladder reveals petechial hemorrhages or glomeraulations in 90% of patients.

The term “male erectile dysfunction” refers a disorder characterized by an inability to achieve and/or maintain an penile erection for satisfactory sexual performance.

The term “outlet obstruction” refers to disease states including, but not limited to, benign prostatic hyperplasia (BPH), urethral stricture disease, tumors, etc. Outlet obstruction can be further defined as obstructive (e.g., low flow rates, difficulty initiating urination, etc.) or irritative (e.g., urgency, suprapubic pain, etc.).

The term “outlet insufficiency” refers to urethral hypermobility or intrinsic sphincteric deficiency and is symptomatically manifested as stress incontinence.

The terms “overactive bladder” or “detrusor hyperactivity” refer to symptoms which manifest as urgency, frequency, and/or incontinence episodes. These symptoms can be caused by changes in bladder capacity, micturition threshold, unstable bladder contractions, and/or sphincteric spasticity. Hyperreflexia, outlet obstruction, outlet insufficiency, and pelvic hypersensitivity can also be idiopathic for this disease state.

The term “pelvic hypersensitivity” refers to pelvic pain, incontinence, prostadynia, prostatis, vulvadynia, urethritis, orchidalgia, etc. Pelvic hypersensitivity can be manifested as pain or discomfort in the pelvic region and also usually includes symptoms of overactive bladder defined above.

The term “soluble epoxide hydrolase inhibitor” refers to a compound that inhibits soluble epoxide hydrolase with an IC50 of less than 1 μM, preferably less than 100 nM. IC50s may be determined by standard methods. One particular method is a calorimetric assay as described in Example 3.

The term “subject” refers to mammals and non-mammals. Examples of mammals include, but are not limited to, any member of the Mammalia class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. The term does not denote a particular age or gender

The term “treating” or “treatment of” a disease state includes: 1) preventing the disease state, i.e. causing the clinical symptoms of the disease state not to develop in a subject that may be exposed to or predisposed to the disease state, but does not yet experience or display symptoms of the disease state; 2) inhibiting the disease state, i.e., arresting the development of the disease state or its clinical symptoms; 3) or relieving the disease state, i.e., causing temporary or permanent regression of the disease state or its clinical symptoms.

Chemical structures shown herein were prepared using ISIS® version 2.2. Any open valency appearing on a carbon, oxygen or nitrogen atom in the structures herein indicates the presence of a hydrogen.

### The Invention

The present invention is based on the discovery that soluble epoxide hydrolase plays an important role in regulating contractions of the bladder detrusor smooth muscle. Differential gene expression studies using Affymetrix GeneChips (Example 1) and Quantitative Reverse Transcriptase (qRT)-PCR (Example 2) were conducted in which messenger RNA (mRNA) levels from bladders between SHR and WKY rats were compared. Soluble epoxide hydrolase was identified as being the most highly up-regulated gene in SHR bladders relative to WKY bladders, suggesting that increased levels or activity of sEH contribute to the observed symptoms of bladder overactivity in the SHR such as high micturition frequency and low bladder volume. Therefore, inhibition of sEH should have beneficial effects in the treatment of disease states of the urinary tract such as overactive bladder. Furthermore, an increase in the level or activity of sEH in a urine sample or bladder tissue from a subject would suggest that the subject may be at risk for a genitourinary disorder.

A number of classes of sEH inhibitors have been identified. WO00/23060, which is incorporated herein by reference, discloses the 1-(4-aminophenyl)pyrazole class of compounds which inhibit sEH with submicromolar IC50s and display anti-inflammatory activities. These compounds have structures as represented by Formula 1.

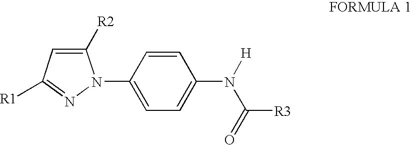

where R1 is 3-pyridinyl, MeOCH₂, I—Pr, Et, CF₃, or Me; R2 is Et, CF₃, I—Pr, 2-oxazolidinyl, or Me; R3 is 3-pyridinyl, 3,5-dimethyloxazol-4-yl, or 2-chloropyridinin-4-yl. A representative member from this series, Compound 1, (N-[4-(5-ethyl-3-pyridin-3-yl-pyrazol-1-yl)-phenyl]-nicotinamide) was used for experiments described below.

Other classes of sEH inhibitors include the chalcone oxide derivatives (Mullin and Hammock, Arch. Biochem. Biophys., 216, 423-429 (1982); Miyamoto, et al, Arch. Biochem. Biophys., 254, 203-213 (1987)) and various trans-3-phenyglycidols (Dietze, et al, Biochem Pharm. 42, 1163-1175 (1991); Dietze et al, Comp. Biochem. Physiol. B, 104, 309-314 (1993)). More recently, Hammock et al. have disclosed as series of 1,3-disubstituted ureas, carbamates and amides with nanomolar IC50 values (U.S. Pat. No. 6,531,506; Morisseau et al, Biochem. Pharmacology 63, 1599-1608 (2002), both of which are incorporated herein by reference). QSAR modelling analysis of 348 of these compounds has also been published (McElroy et al, J. Med. Chem. 46, 1066-1080 (2003)). The structure of these compounds are represented by Formula 2

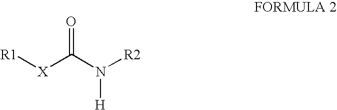

where X is NH, O, or CH₂, R1 and R2 are alkyl or aryl groups. Representative compounds from this series of compounds include, N-cyclohexyl-N-4-chlorophenylurea, N,N′-bis(3,4-dichlorophenyl)urea, and N-cyclopentyl-N′-dodecylurea.

The effect of the sEH inhibitor, Compound 1, on micturition was tested using anesthetized Spontaneously Hypertensive Rats (Example 4). Intravenous infusion of the inhibitor resulted in a dose-dependent reduction in the both the frequency and the amplitude of the involuntary contractions of the bladder detrusor muscle, confirming the utility of an sEH inhibitor for the treatment of the symptoms of an overactive bladder. Because inhibition of sEH should result in the accumulation of its substrate(s), the effects of 14,15-EET on isolated bladder tissue was examined (Example 5). These studies showed that 14,15-EET relaxes bladder smooth muscle which had been stimulated by low frequency electric fields, associated with purinergic mechanisms. This relaxation effect was specific for 14,15-EET in that 8,9-EET, 11,12-EET and 14,15-DHET all showed no effects.

### Pharmaceutical Formulations and Modes of Administration

The methods described herein use pharmaceutical compositions comprising the molecules described above, together with one or more pharmaceutically acceptable excipients or vehicles, and optionally other therapeutic and/or prophylactic ingredients. Such excipients include liquids such as water, saline, glycerol, polyethyleneglycol, hyaluronic acid, ethanol, etc. Suitable excipients for nonliquid formulations are also known to those of skill in the art. Pharmaceutically acceptable salts can be used in the compositions of the present invention and include, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients and salts is available in Remington's Pharmaceutical Sciences, 18th Edition (Easton, Pa.: Mack Publishing Company, 1990).

Additionally, auxiliary substances, such as wetting or emulsifying agents, biological buffering substances, surfactants, and the like, may be present in such vehicles. A biological buffer can be virtually any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, and the like

Depending on the intended mode of administration, the pharmaceutical compositions may be in the form of solid, semi-solid or liquid dosage forms, such as, for example, tablets, suppositories, pills, capsules, powders, liquids, suspensions, creams, ointments, lotions or the like, preferably in unit dosage form suitable for single administration of a precise dosage. The compositions will include an effective amount of the selected drug in combination with a pharmaceutically acceptable carrier and, in addition, may include other pharmaceutical agents, adjuvants, diluents, buffers, etc.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, etc., an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. Actual methods of preparing such dosage forms are known, or will be apparent, to those skilled in this art; for example, see Remington's Pharmaceutical Sciences, referenced above.

For oral administration, the composition will generally take the form of a tablet, capsule, a softgel capsule or may be an aqueous or nonaqueous solution, suspension or syrup. Tablets and capsules are preferred oral administration forms. Tablets and capsules for oral use will generally include one or more commonly used carriers such as lactose and corn starch. Lubricating agents, such as magnesium stearate, are also typically added. When liquid suspensions are used, the active agent may be combined with emulsifying and suspending agents. If desired, flavoring, coloring and/or sweetening agents may be added as well. Other optional components for incorporation into an oral formulation herein include, but are not limited to, preservatives, suspending agents, thickening agents, and the like.

Parenteral formulations can be prepared in conventional forms, either as liquid solutions or suspensions, solid forms suitable for solubilization or suspension in liquid prior to injection, or as emulsions. Preferably, sterile injectable suspensions are formulated according to techniques known in the art using suitable carriers, dispersing or wetting agents and suspending agents. The sterile injectable formulation may also be a sterile injectable solution or a suspension in a nontoxic parenterally acceptable diluent or solvent. Among the acceptable vehicles and solvents that may be employed are water, Ringer's solution and isotonic sodium chloride solution. In addition, sterile, fixed oils, fatty esters or polyols are conventionally employed as solvents or suspending media. In addition, parenteral administration may involve the use of a slow release or sustained release system such that a constant level of dosage is maintained.

Alternatively, the pharmaceutical compositions of the invention may be administered in the form of suppositories for rectal administration. These can be prepared by mixing the agent with a suitable nonirritating excipient which is solid at room temperature but liquid at the rectal temperature and therefore will melt in the rectum to release the drug. Such materials include cocoa butter, beeswax and polyethylene glycols. The pharmaceuticals compositions of the invention can also be formulated for vaginal administration. Pessaries, tampons, creams, gels, pastes, foams or sprays containing in addition to the active ingredient such carriers are known in the art to be appropriate.

The pharmaceutical compositions of the invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, propellants such as fluorocarbons or nitrogen, and/or other conventional solubilizing or dispersing agents.

Preferred formulations for topical drug delivery are ointments and creams. Ointments are semisolid preparations which are typically based on petrolatum or other petroleum derivatives. Creams containing the selected active agent, are, as known in the art, viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

Formulations for buccal administration include tablets, lozenges, gels and the like. Alternatively, buccal administration can be effected using a transmucosal delivery system as known to those skilled in the art. The compounds of the invention may also be delivered through the skin or muscosal tissue using conventional transdermal drug delivery systems, i.e., transdermal “patches” wherein the agent is typically contained within a laminated structure that serves as a drug delivery device to be affixed to the body surface. In such a structure, the drug composition is typically contained in a layer, or “reservoir,” underlying an upper backing layer. The laminated device may contain a single reservoir, or it may contain multiple reservoirs. In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or gel reservoir, or may take some other form. The backing layer in these laminates, which serves as the upper surface of the device, functions as the primary structural element of the laminated structure and provides the device with much of its flexibility. The material selected for the backing layer should be substantially impermeable to the active agent and any other materials that are present.

A pharmaceutically or therapeutically effective amount of the composition will be delivered to the subject. The precise effective amount will vary from subject to subject and will depend upon the species, age, the subject's size and health, the nature and extent of the condition being treated, recommendations of the treating physician, and the therapeutics or combination of therapeutics selected for administration. Thus, the effective amount for a given situation can be determined by routine experimentation. For purposes of the present invention, generally a therapeutic amount will be in the range of about 0.05 mg/kg to about 40 mg/kg body weight, more preferably about 0.5 mg/kg to about 20 mg/kg, in at least one dose. In larger mammals the indicated daily dosage can be from about 1 mg to 100 mg, one or more times per day, more preferably in the range of about 10 mg to 50 mg. The subject may be administered as many doses as is required to reduce and/or alleviate the signs, symptoms, or causes of the disorder in question, or bring about any other desired alteration of a biological system.

The delivery of polynucleotides, e.g., for delivering soluble epoxide hydrolase antisense oligonucleotides, can be achieved using any of the formulations described above, or by using recombinant expression vectors, with or without carrier viruses or particles. Such methods are well known in the art. See, e.g., U.S. Pat. Nos. 6,214,804; 6,147,055; 5,703,055; 5,589,466; 5,580,859; Slater et al. (1998) J. Allergy Clin. Immunol. 102:469-475. For example, delivery of polynucleotide sequences can be achieved using various viral vectors, including retrovirus and adeno-associated virus vectors. See, e.g., Miller A. D. (1990) Blood 76:271; and Uckert and Walther (1994) Pharmacol. Ther. 63:323-347. Vectors which can be utilized for antisense gene therapy include, but are not limited to, adenoviruses, herpes viruses, vaccinia, or, preferably, RNA viruses such as retroviruses. Other gene delivery mechanisms that can be used for delivery of polynucleotide sequences to target cells include colloidal dispersion and liposome-derived systems, artificial viral envelopes, and other systems available to one of skill in the art. See, e.g., Rossi, J. J. (1995) Br. Med. Bull. 51:217-225; Morris et al. (1997) Nucl. Acids Res. 25:2730-2736; and Boado et al. (1998) J. Pharm. Sci. 87:1308-1315. For example, delivery systems can make use of macromolecule complexes, nanocapsules, microspheres, beads, and lipid-based systems including oil-in-water emulsions, micelles, mixed micelles, and liposomes.

All patents, patent applications, and publications mentioned herein, whether supra or intra, are each incorporated by reference in its entirety. The broad scope of this invention is best understood with reference to the following examples, which are not intended to limit the invention to the specific embodiments described below.

### EXAMPLES

The following preparations and examples are given to enable those skilled in the art to more clearly understand and to practice the present invention. They should not be considered as limiting the scope of the invention, but merely as being illustrative and representative thereof.

### Example 1

### Gene Expression Profiling of Spontaneously Hypertensive Rat Bladders

Affymetrix GeneChip profiling was performed on the whole bladders of 6 Spontaneously Hypertensive Rats (SHR) and 6 Wistar-Kyoto Rats (WKY). Differential gene expression between SHR and WKY rat bladders were analyzed with the intent to identify potential genes of interest for overactive bladder (OAB).

Total RNA was isolated from whole bladders using the Trizol method. The isolated total RNA was quantitated by spectrophotometric readings at O.D. 260 and qualified by agarose gel electrophoresis and the Agilent BioAnalyzer RNA 6000 Assay.

First strand and second strand cDNA was generated from 10 μg total RNA using AMV reverse transcriptase and the “cDNA Synthesis System” kit components from Roche Applied Science (cat #1117831). To generate the cDNA, an oligo dT (24mer)-T7 primer was used to prime the mRNA for the first strand synthesis. After the second strand cDNA synthesis step, the sample was phenol/chloroform extracted and salt precipitated with ammonium acetate and ethanol. The pellet was resuspended in DEPC-treated water.

The ENZO Diagnostics “BioArray High Yield RNA Transcript Labeling Kit (T7 RNA Polymerase)” (cat #42655-10) was then used for the in vitro transcription step utilizing one-half of the previously synthesized cDNA. During this T7 RNA polymerase driven in vitro transcription step, biotin-labeled ribonucleotides were incorporated. The reaction was carried out in a volume of 40 μL at 37 C for 6 hours. The samples were then run over Qiagen RNeasy mini-columns to purify the sample of unincorporated nucleotides.

The in vitro transcribed biotin-labeled RNA samples were quantitated and quality checked by the methods described above. 12 μg of the sample was then fragmented in an acetate buffer and brought up in the hybridization cocktail.

10 μg of the sample was hybridized onto the rat Affymetrix U34A chips for 16 hours. The chips were then washed with non-stringent and stringent buffers and stained. The staining process involved a primary stain, streptavidin labeled with phycoerythrin (SAPE), followed by a secondary antibody amplification stain which in turn was followed by a tertiary SAPE stain. Following the staining process, the individual chips were scanned. Soluble epoxide hydrolase, NCBI protein record number P80299, was found to be the most highly upregulated gene on the U34A gene expression array in SHR bladders relative to Wistar-Kyoto bladders in terms of fold expression SHR/WKY.

### Example 2

### TaqMan Real-Time Quantitative Reverse Transcriptase (qRT)-PCR

RNA was prepared from rat bladders as in Example 1 and stored at −80 C until experiments were performed. Real-time quantitative polymerase chain reaction (RT-PCR) analysis (Heid et al., Genome Res. 6, 986-994 (1996)) was used to determine the relative levels of rat and human soluble epoxide hydrolase from total RNA. Prior to amplification, the total RNA samples were DNAse I treated and purified using Qiagen's “Rneasy Mini Kit” according to the manufacturer's instructions (cat. #74104, Qiagen Inc., Valencia, U.S.A.). Reverse transcription and PCR reactions were performed using “One-Step RT-PCR Master Mix Reagents” according to the manufacturer's instructions (cat. #4309169, Applied Biosystems, Foster City, U.S.A.). Rat and human soluble epoxide hydrolase sequence-specific amplification was detected with an increasing fluorescent signal of FAM reporter dye during the amplification cycle. Each sequence specific amplification was done in duplicate. Levels of the different mRNAs were subsequently normalized to an 18S rRNA control (cat. # 4308329, Applied Biosystems). Oligonucleotide primers and TaqMan probes were designed using Primer Express software (Applied Biosystems) and were synthesized by Applied Biosystems.

Forward primer: 5′-GGAGAAAGTCACAGGGACACAGTTT-3′ (SEQ ID NO:1)
reverse primer: 5′-GGAAACCCATGACAGAGGCATATA-3′ (SEQ ID NO:2)
probe: 5′-6FAM-CCAAATGATGTCAGCCATGGGTATGTGA-TAMRA (SEQ ID NO:3)

### Example 3

### Synthesis and Determination of IC50 for Compound 1

Compound 1, (N-[4-(5-ethyl-3-pyridin-3-yl-pyrazol-1-yl)-phenyl]-nicotinamide),

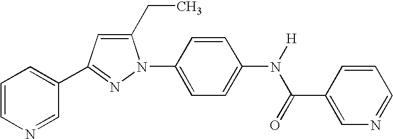

was synthesized as described (WO 00/23060, compound 1). The IC50 was determined with the colorimetric substrate 4-nitrophenyl-(2S,3S)-2,3-epoxy-3 phenylpropyl carbonate as substrate as described by Dietze et al Anal. Biochem. 216, 176-187 (1994)). The IC50 was found to be 0.084+/−0.002 micromolar when assayed with 100 nanomolar human soluble epoxide hydrolase expressed (Beetham et al. Arch. Biochem. Biophys. 305, 197-201 (1993)) and purified as described by Wixtrom et al. (Anal. Biochem. 169, 71-80 (1994)) at 40 micromolar substrate concentration and 30° C.

### Example 4

### Inhibition of Soluble Epoxide Hydrolase Activity in Anesthetized Rats

The effect on micturition of inhibition of soluble epoxide hydrolase enzyme activity of this invention in vivo was determined in rats using a modification of the method described in Yoshiyama, M. et al. Brain Research, (1994) March 14, 639(2):300-8.

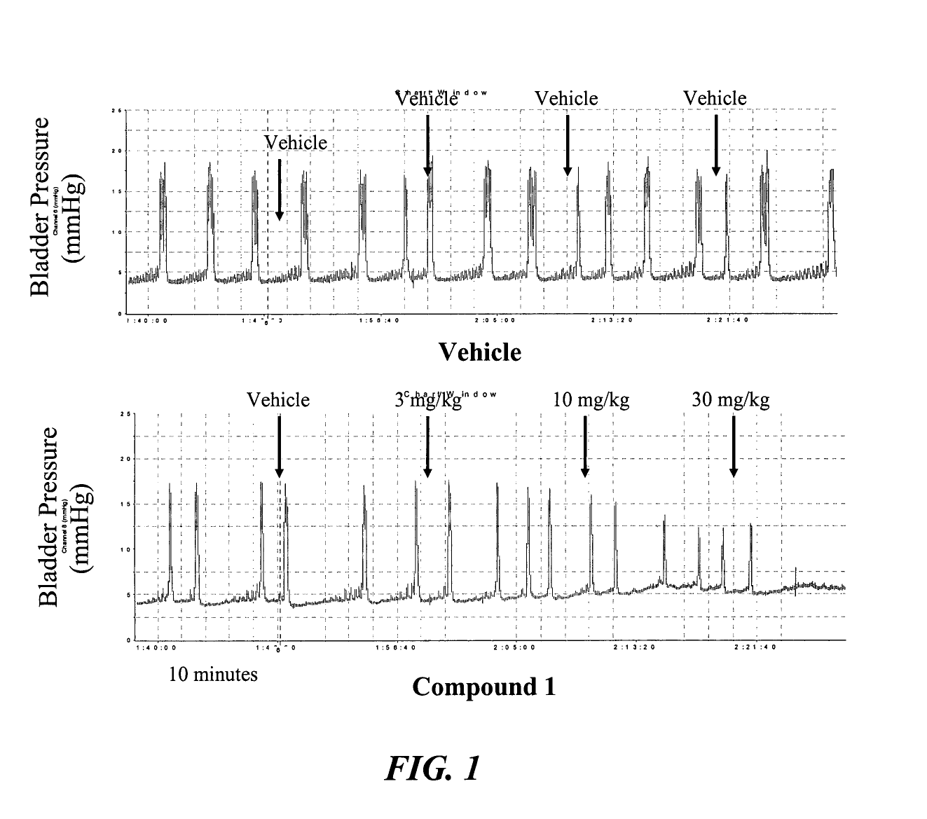

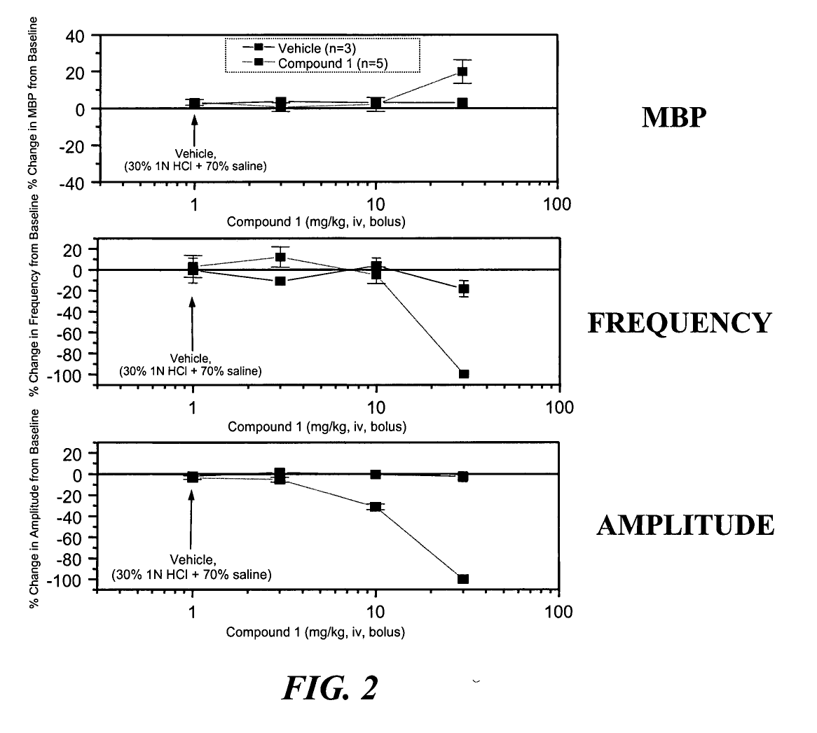

Female Spontaneously Hypertensive Rats (SHRs) were anesthetized with urethane (1.5 g/kg, sc). The trachea was exposed and cannulated with polyethylene (PE)-240 tubing (Becton-Dickinson). The right carotid artery and left femoral vein were cannulated with PE-50 tubing for measurement of blood pressure and administration of drugs, respectively. An incision was made into the lower peritoneal cavity along the linea-alba, exposing the ureters and the urinary bladder. Both ureters were ligated and cut, allowing urine from the kidneys to drain into the abdomen. The bladder was cannulated via the dome with PE-50 tubing and the cannula secured in place with a ligature (3-0 silk suture). The bladder cannula was connected to both a transducer and syringe infusion pump (Harvard Apparatus) via a “Y-connector.” Mean blood pressure and micturition contractions were recorded throughout the experiment using Gould pressure transducers (P23XL) connected to a Gould recorder (Gould 3800) and a Power Lab data acquisition system. After a one-hour stabilization period, saline was infused into the urinary bladder at 0.1 ml/min for a period of 1 hour. At the end of the 1-hour saline infusion, compounds or vehicle were administered intravenously as a cumulative dose-response or single bolus injection. Bladder contraction amplitude and frequency was measured and test compounds were compared to their vehicle time control. The animals were euthanized by a lethal dose of Pentobarbital sodium (Ro100-5534/033), i.v. at the end of the study. The cystometric measures of the effects of Compound 1 in anesthetized SHRs are shown in FIGS. 1 and 2.

### Example 5

### Contraction Studies

Bladder strips from (male/female) Sprague-Dawley (Charles River) rat bladders were mounted in 10 ml tissue baths maintained at 37 C, containing 10 ml of a saline solution consisting of: NaCl (118.5 mM), KCl (4.8 mM), NaHCO₃ (25 mM), KH₂PO₄ (1.2 mM), MgSO₄ (1.2 mM), CaCl₂ (2.5 mM), and glucose (11.0 mM). The bladder strips were aerated with a mixture of 95% O₂ and 5% CO₂. The tissues were initially equilibrated for one h with 1 g. resting weight. The control response to 67 mM KCl was then determined. Electrical field stimulation (EFS) was provided through platinum electrodes of 1.14 cm² surface area located one cm apart on either side of the tissue. Stimulation to the platinum electrodes was provided by a GRASS Medical Instruments (Quincy, Mass.) S88 Square Pulse Stimulator set to deliver 10 V pulses with a 0.5 ms pulse duration in a pulse train of 10 seconds at either 1, 2, 4, or 8 Hz.

What is claimed is:

1. A method of treating a mammalian subject having a disease state associated with a genitourinary disorder comprising administering to the subject an effective amount of a soluble epoxide hydrolase inhibitor.

2. The method of claim 1, wherein the genitourinary disorder is an overactive bladder, outlet obstruction, outlet insufficiency, interstitial cystitis, or pelvic hypersensitivity.

3. The method of claim 1, wherein the genitourinary disorder is an overactive bladder.

4. The method of claim 1, wherein the effective amount of the soluble epoxide hydrolase inhibitor is administered orally.

5. The method of claim 1, wherein the mammalian subject is a human.

6. The method of claim 1, wherein the soluble epoxide hydrolase inhibitor has an IC50 of less than 1 μM.

7. The method of claim 1, wherein the soluble epoxide hydrolase inhibitor is a compound of Formula I 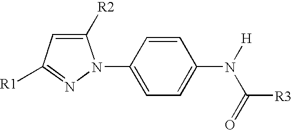
wherein R1 is 3-pyridinyl, MeOCH₂, I—Pr, Et, CF₃, or Me; R2 is Et, CF₃, I—Pr, 2-oxazolidinyl, or Me; R3 is 3-pyridinyl, 3,5-dimethyloxazol-4-yl, or 2-chloropyridinin-4-yl, or a pharmaceutically acceptable salt thereof.

8. The method of claim 1, wherein the soluble epoxide hydrolase inhibitor is N-[4-(5-ethyl-3-pyridin-3-yl-pyrazol-1-yl)-phenyl]-nicotinamide, or a pharmaceutically acceptable salt thereof.

9. The method of claim 1, wherein the soluble epoxide hydrolase inhibitor is a compound of 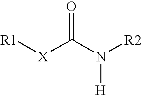
Formula II wherein X is NH, O, or CH₂, R1 and R2 are alkyl or aryl groups, or a pharmaceutically acceptable salt thereof.

10. A method for decreasing bladder contraction frequency and amplitude in a mammalian subject comprising administering to the subject an effective amount of a soluble epoxide hydrolase inhibitor.

11. The method of claim 10, wherein the effective amount of the soluble epoxide hydrolase inhibitor is administered orally.

12. The method of claim 10, wherein the mammalian subject is a human.

13. The method of claim 10, wherein the soluble epoxide hydrolase inhibitor has an IC50 of less than 1 μM.

14. The method of claim 10, wherein the soluble epoxide hydrolase inhibitor is a compound of Formula I 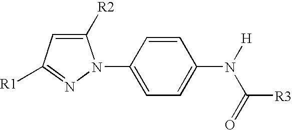
wherein R1 is 3-pyridinyl, MeOCH₂, I—Pr, Et, CF₃, or Me; R2 is Et, CF₃, I—Pr, 2-oxazolidinyl, or Me; R3 is 3-pyridinyl, 3,5-dimethyloxazol-4-yl, or 2-chloropyridinin-4-yl, or a pharmaceutically acceptable salt thereof.

15. The method of claim 10, wherein the soluble epoxide hydrolase inhibitor is N-[4-(5-ethyl-3-pyridin-3-yl-pyrazol-1-yl)-phenyl]-nicotinamide or a pharmaceutically acceptable salt thereof.

16. The method of claim 10, wherein the soluble epoxide hydrolase inhibitor is a compound 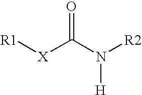
of Formula II wherein X is NH, O, or CH₂, R1 and R2 are alkyl or aryl groups, or a pharmaceutically acceptable salt thereof.

17. A method of identifying compounds that decrease bladder contraction frequency and amplitude in a mammalian subject, the method comprising: a) contacting the compound with soluble epoxide hydrolase and determining whether the compound inhibits soluble epoxide hydrolase and b) testing the compound in a functional assay that measures the effect of the compound on bladder contraction frequency and amplitude.

18. A method of identifying a mammalian subject at risk for a genitourinary disorder, the method comprising assaying for soluble epoxide hydrolase level or activity in a sample from the subject.

19. The method of claim 18, wherein the sample is a bladder tissue or a urine sample.

20. A method of treating a mammalian subject having a disease state associated with a genitourinary disorder comprising administering to the subject an effective amount of a 14,15-EET receptor agonist.

21. The method of claim 20, wherein the genitourinary disorder is an overactive bladder, outlet obstruction, outlet insufficiency, interstitial cystitis, or pelvic hypersensitivity.

22. The method of claim 20, wherein the genitourinary disorder is an overactive bladder.

23. The method of claim 20, wherein the effective amount of the agonist is administered orally.

24. The method of claim 20, wherein the mammalian subject is a human.

25. The method of claim 20, wherein the agonist has an affinity value of less than 100 nM to the 14,15-EET receptor.
